(19) United States
(12) Patent Application Publication
(10) Pub. No.: US 2011/0172305 A1
(43) Pub. Date: Jul. 14, 2011

(54) Endocannabinoids for Enhancing Growth and Development in Infants

(76) Inventors: Ester Fride, Efrat (IL); Ayala Polak-moshe, Kfar-Saba (IL); David Branski, Jerusalem (IL); Shimon Ben-Shabat, Jerusalem (IL)

(21) Appl. No.: 12/681,273

(22) PCT Filed: Oct. 2, 2008
(86) PCT No.: PCT/IL08/01314

§ 371 Date: Nov. 10, 2010

Related U.S. Application Data
(60) Provisional application No. 60/960,512, filed on Oct. 2, 2007.

(51) Int. Cl. A61K 31/22; A61K 31/16; A61K 31/216; A61K 31/221; C07C 229/30; C07C 233/20; C07C 59/42; C07C 233/22; A61P 25/00; A61P 3/02; A23L 1/30

(52) U.S. Cl. 514/549; 514/627; 554/103; 554/35; 554/224; 426/648

(57) ABSTRACT

The present invention relates to a method for promoting infant feeding, growth or development comprising administering to an infant a formula or a pharmaceutical composition comprising an endocannabinoid in an amount sufficient to promote feeding, growth or development. The present invention also relates to an infant formula comprising an enhanced amount of an endocannabinoid. The infant formula of the invention may be in a powder form or in a liquid form. The infant formula or the pharmaceutical composition may further comprise an endocannabinoid-promoting compound.

### FIELD OF THE INVENTION

This invention relates to the promotion of feeding, growth, and development in infants, by the use of endocannabinoids (ECs) and their derivatives. In particular, the to present invention concerns infant formulas comprising enhanced levels of ECs and methods for their preparation.

### BACKGROUND OF THE INVENTION

The major active molecule in the marijuana (Cannabis sativa) plant, Δ⁹-tetrahydrocannabinol (THC), activates at least two specific receptors denoted CB₁ and CB₂. Since 1992, several endogenous ligands for these receptors have been isolated from brain and peripheral tissue, entitled the “endocannabinoids” (ECs; see for example (Fride and Gobshtis, 2007).

The endocannabinoid-receptor system has many physiological roles including the regulation of memory, management of pain and inflammatory processes, immune regulation as well as feeding and appetite. The first ECs to be isolated and the most extensively studied are “anandamide” (AEA) and 2-arachidonoyl glycerol (2-AG).

These ECs, similarly to the marihuana plant-derived THC, enhance appetite and food intake. THC (dronabinol) has been clinically used to combat reduction in appetite and wasting in AIDS and cancer patients. Cannabinoid treatment was also suggested for appetite enhancement and wasting in other conditions, i.e. cystic fibrosis, Alzheimer disease and anorexia (for review see Fride et al., 2005).

EC, specifically 2AG, were detected in human maternal milk.

In addition, it has been proposed that EC and specifically 2AG in the newborn's brain comprises a major stimulus to initiate milk intake (Fride et al., 2001).

None of these publications suggests the administration of EC to infants as a means to increase food intake and subsequent growth and development.

### SUMMARY OF THE INVENTION

The present invention is based on the notion that in infants with compromised feeding and/or growth, a supplement of endocannabinoids, e.g. 2-arachidonoyl glycerol will enhance growth. One way of supplementing EC to infants is via an infant formula which contains increased levels of endocannabinoids or endocannabinoid-promoting compounds. Such a formula may be useful to promote appetite and weight gain in infants.

Accordingly, the present invention provides by a first of its aspects a method for promoting infant feeding, growth or development comprising administering to an infant in need thereof a formula or a pharmaceutical composition comprising an endocannabinoid in an amount sufficient to promote feeding, growth or development. In certain embodiments, said infant formula administered to the infant is sufficient to provide the infant with at least about 0.04 mg/kg/day, 0.05 mg/kg/day, 0.06 mg/kg/day, 0.07 mg/kg/day, or 0.1 mg/kg/day of endocannabinoids.

By another aspect, the present invention provides an infant formula comprising an enhanced amount of an endocannabinoid. According to one embodiment, the infant formula of the invention comprises fat, protein, carbohydrate, vitamins, minerals, trace elements, and at least about 0.2 mg, 0.3 mg, 0.4 mg, 0.5 mg, or 1 mg of endocannabinoids per 1 liter of liquid formula.

The infant formula of the invention may be in a powder form or in a liquid form. In one embodiment, the infant formula is in powder form and is hydrated prior to consumption so as to assume a liquid form.

By yet another aspect, the present invention further provides a pharmaceutical composition comprising an endocannabinoid for promoting infant, child, or adolescent feeding, growth or development.

The infant formula or the pharmaceutical composition may further comprise an endocannabinoid-promoting compound. In one embodiment the endocannabinoid-promoting compound is a fatty acid-related molecule (e.g. 2-palmytoyl glycerol, or 2-linoleoyl-glycerol (2-LINO-GL)). Without wishing to be bound by theory, such a molecule when provided with an endocannabinoid may potentiate the endocannabinoid activity, for example by preventing breakdown and/or by potentiating receptor binding. According to one embodiment, the infant formula of the invention comprises at least about 0.05 mg, 0.1 mg, 0.2 mg, 0.5 mg, or 1 mg of endocannabinoid-promoting compounds per 1 liter of liquid formula.

In accordance with the present invention, the endocannabinoid in the infant formula or in the pharmaceutical composition is selected from the group consisting of anandamide, 2-arachidonoyl glycerol (2AG), noladin ether, N-arachidonoylglycerol dopamine (NADA) and virodhamine (which are considered partial agonists/antagonists of CB₁ receptors).

In another aspect, the present invention provides an infant formula comprising an endocannabinoid-promoting compound for promoting infant feeding, growth, or development.

In another aspect, the present invention provides a pharmaceutical composition comprising an endocannabinoid-promoting compound for promoting infant, child, or adolescent feeding, growth or development.

In one embodiment, the endocannabinoid promoting compound is an inhibitor of an endocannabinoid degrading enzyme or a reuptake inhibitor.

In one embodiment, the endocannabinoid-promoting compound is 2-palmytoyl glycerol or 2-linoleoyl-glycerol (2-LINO-GL).

The present invention also concerns a method for promoting infant, child or adolescent feeding, growth or development comprising administering to an infant, a child or an adolescent in need thereof a pharmaceutical composition comprising an endocannabinoid promoting compound in an amount sufficient to promote feeding, growth or development.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 is a graph showing the increase in body weight (in grams) with days of age in four groups of tested mice: Vehicle small—mice grown in a small litter treated with vehicle; 2-AG small—mice grown in a small litter treated with 2-AG; Vehicle large—mice grown in a large litter treated with vehicle; 2-AG large—mice grown in a large litter treated with 2-AG. The graph demonstrates improvement of the weight curve in 2-AG treated malnourished (i.e. grown in large) litters.

FIG. 2 is a graph showing the body weight (grams) of three months old mice which were grown as puppies in either large or small litters and treated as neonates with vehicle or 2-AG. The graph demonstrates that neonatal 2-AG has a permanent weight enhancing effect on malnourished litters.

FIG. 3A is a graph showing the behavior of mice in a “Forced swim test”, specifically the time of staying immobile (in seconds) for two groups of mice. One treated at infancy with 2-AG and one with vehicle. The graph demonstrates antidepressant effect at adulthood (as measured by shorter immobility periods) of 2-AG administered to neonates at the first 5 days of life.

FIG. 3B is a graph showing the number of squares crossed by 2-AG-treated mice or mice without such treatment (control) in correlation with time (min), as a measure of motor activity of the mice.

FIG. 4 is a graph showing nipple attachment in SR141716 (Rimonabant)—treated pups (ICR and Sabra, denoted as SR-ICR and SR-Sabra) or without treatment (control, denoted as vehicle-ICR and vehicle-Sabra). The strength of nipple attachment was scored ‘0’ where no holding onto nipple was observed, ‘1’ where weakly holding on to nipple was observed, or ‘2’ where the pup was firmly attached to nipple.

FIG. 5A is a graph scoring food intake by measuring the milkband size after one session of “lapping”. SR+lap: SR-treated pups which were allowed to lick from a dish; SR no lap—SR-treated pups which were not allowed to lick from a dish; Veh+lap: control vehicle-treated pups which were allowed to lick from a dish; Veh no lap—control vehicle-treated pups which were not allowed to lick from a dish. The graph demonstrates that SR-treated pups can ingest by ‘lapping’ when no sucking is required.

FIG. 5B is a graph demonstrating weight gain in grams (g) after one session of “lapping”. SR+lap: SR-treated pups which were allowed to lick from a dish; SR no lap—SR-treated pups which were not allowed to lick from a dish; Veh+lap: control vehicle-treated pups which were allowed to lick from a dish; Veh no lap—control vehicle-treated pups which were not allowed to lick from a dish.

FIG. 6 is a graph showing the number of squares crossed by mice treated with rimonabant at birth or mice without such treatment (vehicle) as a function of time (min), as a measure of motor activity.

FIG. 7 is a graph showing the amount of prepulse inhibition calculated as % PPI in mice treated with rimonabant at birth (SR) or without treatment (vehicle) as a function of background noise (db). The graph is demonstrating impaired sensorimotor gating in the prepulse inhibition of the startle response assay in mice treated with rimonabant at birth.

FIG. 8A is a graph showing the increase in body weight (in grams) with days of age in four groups of tested mice: SR Brain—mouse pups injected with SR (4.5 μg) directly into the brain at 2 mm depth; VEH Brain—mouse pups treated with vehicle; SR s.c.—mouse pups injected subcutaneously (sc) with 20 mg/kg SR; VEH s.c.—mouse pups treated subcutaneously with vehicle.

FIG. 8B is a graph showing the increase in body weight (in grams) with days of age in four groups of tested mice: SR Brain—mouse pups injected with SR (22.5 μg) directly into the brain at 2 mm depth; VEH Brain—mouse pups treated with vehicle; SR s.c.—mouse pups injected subcutaneously (sc) with 40 mg/kg SR; VEH s.c.—mouse pups treated subcutaneously with vehicle.

FIG. 8C is a graph showing the increase in body weight (in grams) with days of age in four groups of tested mice: SR Brain—mouse pups injected with SR (9 μg) directly into the brain at 3 mm depth; VEH Brain—mouse pups treated with vehicle; SR s.c.—mouse pups injected subcutaneously (sc) with SR; VEH s.c.—mouse pups treated subcutaneously with vehicle.

FIG. 8D is a graph showing the increase in body weight (in grams) with days of age in four groups of tested mice: SR Brain—mouse pups injected with SR (4.5 μg) directly into the brain at 3 mm depth; VEH Brain—mouse pups treated with vehicle; SR s.c.—mouse pups injected subcutaneously (sc) with SR; VEH s.c.—mouse pups treated subcutaneously with vehicle.

FIG. 8E is a graph showing the increase in body weight (in grams) with days of age in four groups of tested mice: SR Brain—mouse pups injected with SR (22.5 μg) directly into the brain at 3 mm depth; VEH Brain—mouse pups treated with vehicle; SR s.c.—mouse pups injected subcutaneously (sc) with 40 mg/kg SR; VEH s.c.—mouse pups treated subcutaneously with vehicle.

FIG. 9A is a graph showing an increase in body weight of Spiny mice and Lab (ICR) mice with days of age.

FIG. 9B is a graph showing the body weight of Spiny mice treated with SR141716 (rimonabant) or without treatment (control) mice with days of age.

FIG. 9C is a graph showing the percent survival of Spiny mice treated with SR141716 (rimonabant) or without treatment (control) mice with days of age.

### DETAILED DESCRIPTION OF EMBODIMENTS

The present invention concerns compositions, and specifically, infant formulas comprising an enhanced amount of EC.

### TERMS AND DEFINITIONS

The terms “infant” and “baby” are used herein as interchangeable terms and refer to individuals not more than about one year of age, and includes infants from 0 to about 4 months of age, infants from about 4 to about 8 months of age, infants from about 8 to about 12 months of age, low birth weight infants at less than 2,500 grams at birth, preterm infants born at less than about 36 weeks gestational age, typically from about 26 weeks to about 34 weeks gestational age, and infants suffering from reduced weight gain and/or failure-to-thrive.

“Failure-to-thrive” (FTT) is defined as an abnormally low weight arid/or height for age and occurs in 1.5-4% of the children. FTT has been divided into organic (OFTT) and nonorganic (NOFTT) entities. Recent research points to NOFTT (comprising about ⅔ of the FTT cases) as a mild neurodevelopmental disorder or pathophysiology in which an oral-motor defect plays a central role and which may be the result of an as of yet, undefined biological vulnerability. The nature of such vulnerability is unknown.

The term “infant formula” as used herein refers to a nutritional composition which is designed for infants to contain sufficient fat, protein, carbohydrate, minerals and vitamins to potentially serve as the sole source of nutrition when provided in sufficient quantity. The infant formula may be based on various sources, e.g. milk-based, soy-based (vegetarian) or semi-elemental casein-hydrolysate. The infant formula may be ready to feed or in a powder form.

The term “ready to feed” as used herein refers to infant formulas in a liquid form suitable for administration to an infant, including reconstituted powders, diluted concentrates, and manufactured liquids.

The term “enteric coating” as used herein relates to a barrier typically applied to oral intakes that is capable of controling the later particular absorbance location in the digestive system. Enteric coatings prevent release of medication before it reaches the small intestine.

As used herein the term “about” refers to +/−10%

As used herein, all percentages, parts and ratios are by weight of the total composition, unless otherwise specified.

Infant formulas have evolved considerably since their first commercial introduction by Justus von Liebig in 1867. Infant formula developers continuously aimed, inter alia, at mimicking natural breast milk by providing duplicate supplemental caloric value, and proteins similar to those proteins found in natural maternal milk.

Development of infant formulas further aimed at providing infant formula characterized by taste of breast milk.

It is known that weight gain is an important factor in the baby's health, attributing among others to its resistance to infection and FTT.

The present invention is directed to infant formula which contains increased levels of endocannabinoids and/or endocannabinoid promoting compounds which are useful to promote appetite and weight gain. Infant suffering from compromised feeding and/or growth can be fed with infant formula of the present invention in order to enhance growth or increase food intake.

Moreover, the present invention is also directed to pharmaceutical compositions comprising EC or EC promoting compounds for enhancing feeding growth and development in infants, children or adolescents.

The present invention also contemplates the use of EC for enhancing appetite and feeding in “at risk” patient populations, such as AIDS and cancer patients suffering from reduction in appetite and wasting, as well as geriatric patients, or anorexia patients.

### Endocannbinoids

Endocannbinoids (ECs) are endogenous ligands for CB₁ and CB₂ receptors and are naturally produced in the bodies of humans and other animals. EC, specifically 2AG, were detected in human maternal milk. As shown in Fride et al 2001, bovine maternal milk contains between 1.0 and 2.4 μg EC per gram extracted lipids, and human maternal milk contains 6.4-8.7 μg EC per gram extracted lipids.

ECs are associated with many physiological roles including the regulation of memory, management of pain and inflammatory processes, immune regulation as well as feeding and appetite. ECs include anandamide (AEA), 2-arachidonoyl glycerol (2-AG), noladin ether, N-arachidonoylglycerol dopamine (NADA) and virodhamine. As well as peripherally restricted cannabinoid-based compounds such as those described U.S. Pat. No. 6,864,291 (Fride et al). Peripherally restricted cannabinoid-based compounds are endocannabinoids which act exclusively in the periphery (outside the central nervous system) thus preventing central (psychological) side effects. Non-limiting examples of peripherally restricted cannabinoid-based compounds are (+)-cannabidiol-DMH (DMH-1,1-dimethylheptyl-), (+)-7-OH-cannabidiol, (+)-7-COOH-cannabidiol and (+)-7-COOH-cannabidiol-DMH (Fride et al., 2004).

The advantage of using endocannabinoids as opposed to using THC is their origin as mammalian (human) endogenous molecules, and hence they are suitable for human use and have reduced detrimental effects on the human body.

Any source of EC is suitable for use herein provided that such a source is suitable for use in infant formula and is compatible with the other selected ingredients in the formula.

The present invention also contemplates the addition of other CB1 or CB2 agonists, for example synthetic cannabionids e.g. ACEA or JWH015, or plant derived cannabinoids such as the prototype Δ⁹-THC.

### Infant Formulations

In one embodiment of the invention, endocannabinoid-promoting compounds are administered as an integral part of an infant formula.

Accordingly, the present invention provides by one of its aspects, a method for promoting infant feeding, growth or development comprising administering to an infant in need thereof a formula comprising an endocannabinoid in an amount sufficient to promote feeding, growth or development.

The infant formula of the invention comprises fat, protein, carbohydrate, vitamins, minerals, trace elements, and at least about 0.2 mg, 0.3 mg, 0.4 mg, 0.5 mg, or 1 mg of an endocannabinoid per 1 liter of liquid formula.

In one embodiment, said infant formula administered to the infant is sufficient to provide the infant with at least about 0.04 mg/kg/day, 0.05 mg/kg/day, 0.06 mg/kg/day, 0.07 mg/kg/day, or 0.1 mg/kg/day of endocannabinoids.

In one specific example, the formula of the present invention comprises 0.0417 mg/kg per day of an endocannabinoid, e.g. 2-AG, assuming that an infant weighing 7.5 kg on breastfeeding, will consume 0.3132 mg per day of said endocannabinoid.

In one embodiment, the infant formula of the present invention further comprises endocannabinoid-promoting compounds. In one embodiment, the endocannabinoid-promoting compound is 2-palmytoyl glycerol or 2-linoleoyl-glycerol (2-LINO-GL).

The infant formula of the invention comprises at least about 0.05 mg, 0.1 mg, 0.2 mg, 0.5 mg, or 1 mg of endocannabinoid-promoting compounds per 1 liter of liquid formula.

In another embodiment, the endocannabinoids or endocannabinoid-promoting compounds are stabilized. In yet another embodiment, the endocannabinoids, or endocannabinoid-promoting compounds are coated with enteric coating that preserves the activity until the endocannabinoids or the endocannabinoid-promoting compounds are released in the small intestine.

Infant formula of the present invention can be provided in a form of powder or in liquid form. The infant formula provided in form of powder is hydrated prior to consumption. The infant formula will assume a liquid form by mixing it with liquid such as water. Alternatively, infant formulas can be provided in liquid form in either concentrate or in a non-concentrated form.

Protein sources can be any which are used in the art, and may include, by way of an example, soy protein, whey protein, nonfat milk, casein, hydrolyzed protein, human milk, bovine milk, cows' milk, reduced mineral milk, and amino acids. In some embodiments, the infant formula of the present invention comprises protein of no less than 8 grams, 11 grams, 15.5 grams, 20 grams, 24 grams, 30 grams, or 40 grams per liter.

Lipid sources which can be used in the present invention, by way of non-limiting example, are vegetable oils, such as palm oil, canola oil, high oleic sunflower oil, corn oil, soybean oil, palm olein oil, coconut oil, and medium chain triglyceride oils. Therefore, in some embodiments, the infant formula of the present invention comprises fat of no less than 20 grams, 24 grams, 31 grams, 34 grams, 35.5 grams, 36 grams, or 40 grams per liter.

Infant formulas of the present invention may comprise fatty acids selected from the group consisting of saturated fatty acids, monounsaturated, and polyunsaturated fatty acid. Thus, in particular embodiments, the infant formula of the present invention comprises saturated fatty acids of no less than 13 grams, 15 grams, 17 grams, 20 grams, 24 grams, or 30 grams per liter. Moreover, in some embodiments, the infant formula of the present invention comprises polyunsaturated fatty acids of no less than 0.1 grams, 0.2 grams, 1 gram, 2 grams, 4 grams, 8 grams, 15 grams, or 20 grams per liter. In certain embodiments, the infant formula of the present invention comprises monounsaturated fatty acids of no less than 0.5 grams, 1 grams, 2 gram, 4 grams, 7 grams, 12 grams, 15 grams, or 20 grams per liter.

The person skilled in the art would appreciate the beneficial properties of adding linoleic acid (LA) and in particular a-linoleic acid. The later is typically referred as “ALA content”. ALA content is typically associated with improved visual acuity. ALA is typically found in canola oil and soy oil. Therefore, in some embodiments, the infant formula of the present invention comprises linoleic acid in ranges selected from the following groups: 500 to 1700 mg, 1500 to 3100 mg, 2900 to 4200 mg, 4100 to 6700 mg, 6500 to 8300 mg, 8100-11000, or 10500-13500 mg per liter.

The person skilled in the art would appreciate that carbohydrate sources can be any of the following: solids of corn syrup, glucose polymers, lactose, sucrose, maltodextrins, and starch. In some embodiments, the infant formula comprises carbohydrates which can be derived from various sources such as lactose, and fruit/vegetable sucrose, in particular corn syrup sucrose. The infant formula of the present invention comprises carbohydrates of no less than 30 grams, 35 grams, 40 grams, 50 grams, 60 grams, 70 grams, 80 grams or 90 grams per liter.

Additionally, the infant formula of the present invention may comprise proteolytic enzymes, and in particular carbohydrate degrading enzymes such as but not limited to lactase, sucrase, fructose, lipases, and alpha-amylase, a polysaccharide digestion enzyme.

However it should be appreciated that numerous commercially available infant formulas can be used as a basic formula to which the endocannabinoids or the endocannabinoid-promoting compounds are added. By way of non-limiting examples such commercially available formulas include Similac® and its derivatives (from Abbott Labs, Columbus, Ohio, USA) , Enfamil LIPIL® by Mead Johnson, Evansville, Ind., U.S.A. The present invention also discloses hereinafter particular infant formulas for use in the present invention.

The infant formulas and corresponding methods of the present invention can comprise additional or optional ingredients useful in nutritional formula applications.

The present invention also contemplates use of the EC and EC promoting compounds as food additives.

### Controlled Release

The endocannabinoids or the endocannabinoid-promoting compounds of the present invention can be encapsulated with an enteric coating which enables controlled release in the small intestines. The endocannabinoids can be encapsulated with agent suitable agents that require such controlled release. The endocannabinoids or endocannabinoid-promoting compounds can therefore be lyophilized and packaged with lyophilized or dried formula, so that they would not prematurely be digested until after passage through the intestine.

Several techniques and materials are available for controlled drug delivery in the in the intestine. By way of non-limiting example, biodegradable polymers such as polylactide and polyglycolide are described in U.S. Pat. No. 4,767,628, and U.S. Pat. No. 4,897,268. Proteins can further be used for enteric coating as disclosed in U.S. Pat. No. 4,925,673.

The endocannabinoids and endocannabinoid-promoting compounds of the present invention can be stabilized with materials while are available to the person skilled in the art, such as albumin, casein, sucrose and lactose. As described above, these are elements typically available in infant formulas.

While the endocannabinoids and indeed endocannabinoid-promoting compounds of the present invention which were described above are designed to be included within the infant formula, it may well be used by administering them apart of infant formulas. The endocannabinoids or endocannabinoid-promoting compounds can be provided in a form of concentrate or dried powder. This endocannabinoids and endocannabinoid-promoting compound additive can be administered at the time of feeding, or before, and indeed following feeding. In this embodiment, the endocannabinoid additive can also be packaged in a buffered solution. The buffered solution containing the endocannabinoid formulation can be added as drops according to dosage regime suitable with the infant condition.

### Pharmaceutical Compositions

Herein, we disclose novel pharmaceutical compositions comprising endocannabinoids or endocannabinoid-promoting compounds to enhance feeding, growth or development in infants, children, or adolescents and/or for the treatment of failure to thrive (FTT) in infants.

The “pharmaceutical compositions” in accordance with the present invention comprise at least one active ingredient, or pharmaceutically acceptable salt(s) thereof, and may also contain a pharmaceutically acceptable carrier and optionally other therapeutic ingredients. The active ingredient of the present invention comprises endocannabinoids or endocannabinoid-promoting compounds or endocannabinoid analogs. The term “pharmaceutically acceptable” means that the carrier, diluent, or excipient is compatible with the other ingredients of the formulation and not deleterious to the recipient.

The compositions of the invention include compositions suitable for oral, rectal, parenteral (including subcutaneous, intramuscular and intravenous) or inhalation administration. The most suitable route in any particular case will depend on the nature and severity of the conditions being treated and the nature of the active ingredient(s).

The term “pharmaceutically acceptable salt” is intended to include art-recognized pharmaceutically acceptable salts. These non-toxic salts are usually hydrolyzed under physiological conditions, and include organic and inorganic bases. Examples of salts include sodium, potassium, calcium, ammonium, copper, and aluminum as well as primary, secondary, and tertiary amines, basic ion exchange resins, purines, piperazine, and the like. The term is further intended to include esters of lower hydrocarbon groups, such as methyl, ethyl, and propyl.

Examples of the pharmaceutical carrier may include a diluent(s), or excipient(s) such as but not limited to a filler, expander, disintegrator, surfactant, binder, wetting agent, in or lubricant that are normally used accordance with the form of use of the composition. The pharmaceutical carrier may be suitably selected and used in accordance with the administration route of the composition to be obtained. Examples of the carrier include physiological saline, buffered physiological saline, dextrose, water, glycerol, ethanol, and mixtures thereof.

The pharmaceutical composition of the present invention may be used as a solution formulation. It can also be used as a lyophilized formulation in order to preserve it, which can be used by dissolving it in water or in a buffered solution including physiological saline or the like to prepare it to a suitable concentration just before use.

The pharmaceutical composition of the present invention may be used alone or together with other compounds or medicaments required for the treatment or in order to enhance the effectiveness of the treatments disclosed in the present invention.

The pharmaceutical composition may take any form that is known to those skilled in the art. Typical examples thereof include a solid formulation such as a tablet, pill, powder, powdered drug, fine granule, granule, or capsule. As well as liquid formulation such as an aqueous formulation, ethanol formulation, suspension, fat emulsion, liposome formulation, clathrate such as cyclodextrin, syrup, or an elixir.

Said powders, pills, capsules, and tablets can be prepared using an excipient such as lactose, glucose, sucrose, or mannitol; a disintegrate agent such as starch or sodium alginate; a lubricant such as magnesium stearate or talc; a binder such as polyvinyl alcohol, hydroxypropyl cellulose, or gelatin; a surfactant such as fatty acid ester; a plasticizer such as glycerin, and the like. For preparation of a tablet or a capsule, a pharmaceutical carrier in a solid state is used.

Injectable solutions comprising the pharmaceutical composition of the present invention can be prepared using a carrier comprising a salt solution, a glucose solution or a mixture of salt water and a glucose solution.

The compositions may be presented in unit dosage form and prepared by any of the methods well known in the art of pharmacy. Dosage regimes may be adjusted for the purpose to improving the therapeutic response.

For example, several divided dosages may be administered daily or the dose may be proportionally reduced over time. A person skilled in the art normally may determine the therapeutically effective amount and the appropriate regime.

The “therapeutically effective amount” refers to that amount of the compound being administered sufficient to prevent development of or alleviate to some extent one or more of the symptoms of FTT, defined above, or to promote appetite and weight gain in infants,

For example, suitable dosage ranges of the endocannabinoids or endocannabinoid-promoting compounds in the pharmaceutical compositions, can be determined according to the following non exhaustive criteria: effectiveness of the ingredients contained therein; the route of administration; the properties of the prescription; the characteristics of the symptoms of the subject; and the judgment of the physician in charge.

In general, said suitable dosage may fall, for example, within a range of approximately 0.05 mg 10 mg per 1 kg of the body weight, and preferably within a range of approximately 0.1 mg to 5 mg per 1 kg body weight, and more preferably about 1 mg per 1 kg body weight.

However, a dosage may be altered using conventional experiments for optimization of a dosage that are well known in the art. The aforementioned dosage can be divided for administration once to several times a day. Alternatively, periodic administration once every few days or few weeks can be employed.

The amount of the active ingredient contained in the pharmaceutical composition of the present invention can be appropriately selected from a broad range. In general, a suitable amount may fall within a range of approximately 0.000001 to 75 wt %, preferably approximately 0.0001 to 10 wt %.

Pharmaceutical compositions of the present invention may be used for enhancing feeding, growth and development. As a non-limiting example the pharmaceutical compositions of the invention may be used for treatment of FTT and FTT related syndromes in infants. In particular, it can be used to increase the infant appetite and growth.

Without wishing to be bound by theory, the pharmaceutical compositions of the present invention may comprise endocannabinoids in order to modulate CB1 and CB2 receptor related cellular activities.

The physician in charge may decide, according to the stage, symptoms and other manifestations of the FTT and/or loss of appetite and/or impaired development or growth, whether to increase dosage or decrease dosage of the endocannabinoids or endocannabinoid-promoting compounds.

The pharmaceutical compositions of the present invention may be used as adjuvant therapy. The pharmaceutical compositions may be used to enhance the effectiveness of a primary treatment. Therefore, said pharmaceutical composition may be used together with other compounds or medicaments required to promote appetite and weight gain in infants of other related conditions.

The physician in charge of treatment of FTT and/or loss of appetite and/or impaired development or growth may indeed decide to use the pharmaceutical compositions of the present invention to reduce the dosage of the primary treatment.

### EXAMPLES

### Example 1

Treatment of Malnourished Litters with the Endocannabinoid 2-Arachidonoyl Glycerol (2-AG)

Using an environmental manipulation technique, within 24 hours of birth, half of each mouse litter (ICR strain) was cross-fostered to the dam of another litter born on the same day. The mouse pups of every two litters were divided up between the 2 dams such that one dam reared 4 pups (‘small litter’) and the other dam raised 18-20 pups (large litter). Pups raised in the large litter were expected to suffer from malnutrition due to the crowded environment. Half of each litter received daily injections of the endocannabinoid 2-arachidonoyl glycerol (2-AG) or THC, the major psychoactive molecule in the Cannabis sativa (marihuana) plant for the first 5 days of life. The doses used were 1 or 5 mg/kg THC or 0.1, 1 or 5 mg/kg 2-AG. Body weights were recorded daily. The body weight of pups from the small and the large litters were analyzed by two-way analysis-of-variance.

At adulthood (3 months of age), mice were subjected to the open field assay for motor activity (ambulation and rearing), to the Porsolt forced swim test for anti-depressant-like effects and to the prepulse-inhibited acoustic startle response (PPI) assay which assesses sensorimotor gating.

Motor activity in an Open field: Mice were placed in a transparent open field (30×40 cm, divided into 20 squares of equal size), measuring horizontal (ambulatory) and vertical activity, for 6 minutes (min), by manually scoring the number of squares crossed (see (Fride and Mechoulam, 1993).

Porsolt forced swim test: The forced swimming test was based on previous designs (Harkin et al., 2004; Petit-Demouliere et al., 2005; Treit and Menard, 1998): mice were placed in a 2 liter glass beaker (11 cm diameter) filled with water (24+1° C.) up to 30 cm from the bottom (so that the mouse could not touch the bottom) and 8 cm from the rim (so that the mouse cannot escape). Immobility time (when the animal does not move except for small movements required to float) was recorded by 3 experimenters for 9 min. Immobility at 9 min was analyzed by a t-test, P<0.05.

Results: Neither of the doses of THC had an effect on the body weight curve, on food intake (‘milkbands’) or on body temperature.

In addition, the lowest (0.1 mg/kg) and highest (5 mg/kg) doses of 2-AGhad also no effect on the growth curve (data not shown).

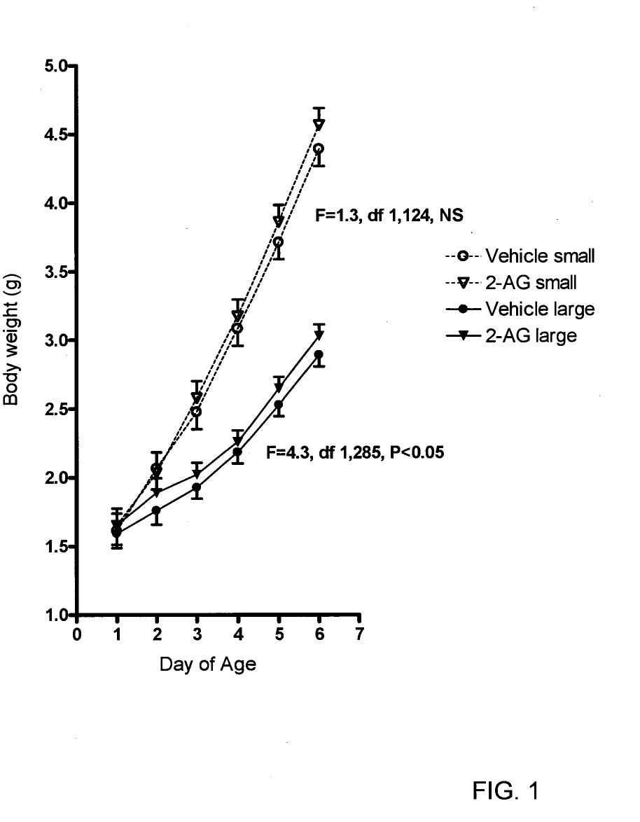

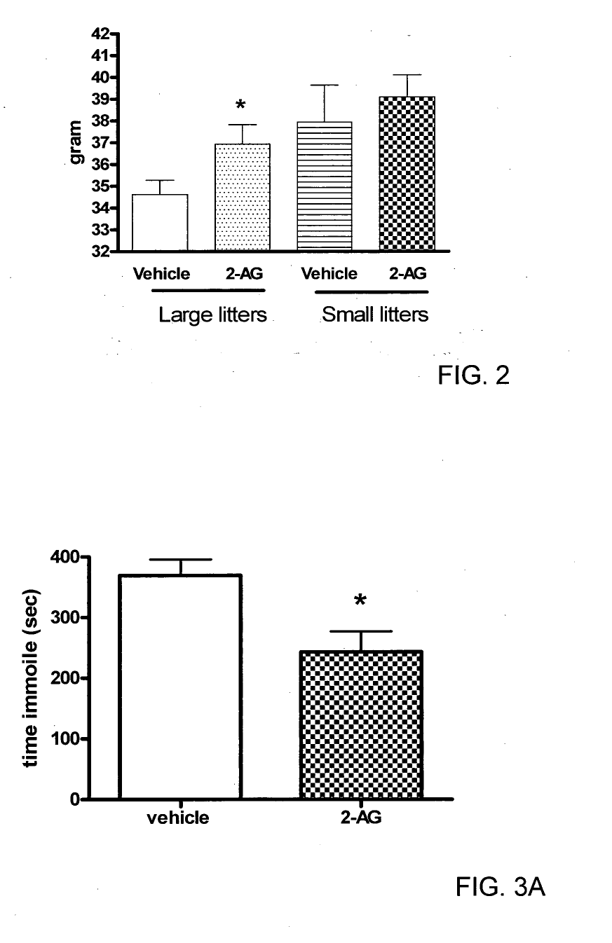

However, it was demonstrated that 1 mg/kg of 2-AG significantly enhanced body weight gain (FIG. 1). Significantly enhanced growth was demonstrated in the malnourished (overcrowded) litters. At 3 months of age, the 2-AG-treated mice still had elevated body weights compared to control mice from large (malnourished) litter. The 2-AG-treated mice in the overcrowded litter (the malnourished mice) did not differ significantly in their body weight from the vehicle-treated mice which were raised in small litters (FIG. 2).

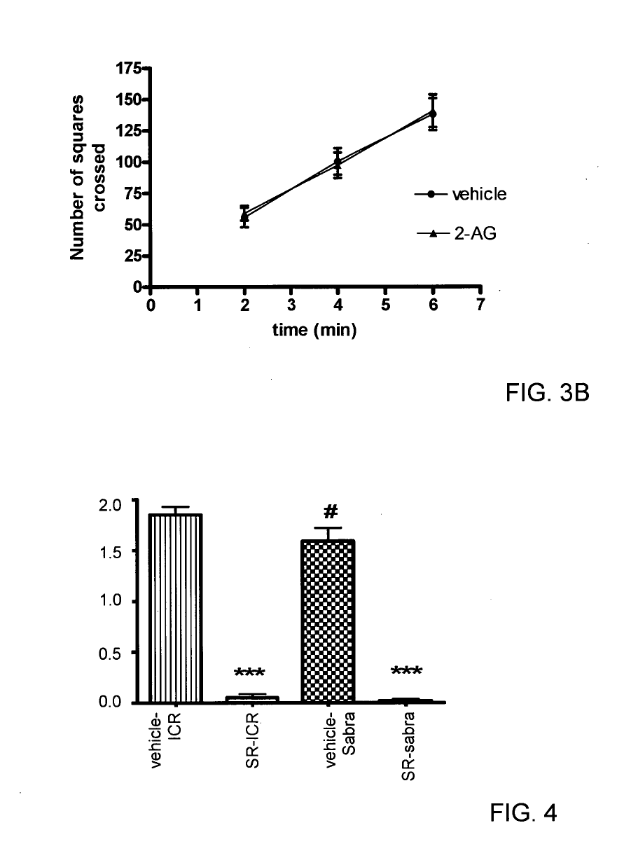

Mice, which were treated with 2-AG in infancy also displayed anti-depressant-like behavior in the Forced swim test (FIG. 3A), suggesting an increased resilience to depression-provoking stimuli.

At 3 months of age, mice which were treated with 2-AG at birth, were tested for motor activity (ambulation) in an open field. Activity was assessed as the number of squares crossed. The performance of 2-AG-treated mice did not differ from that of controls.

This increased resilience to depression-provoking stimuli of mice, which were treated with 2-AG in infancy could not be attributed to enhanced motor activity, since 2-AG-treated mice did not differ from controls in the open field test (FIG. 3B).

### Example 2

### Influence of CB₁ Receptor for Blockade on Pup Feeding and Growth

Pregnant females (Sabra or ICR strain) were housed separately when visibly pregnant (day 12-14 of gestation). Pups were injected within 24 h of birth with 2 vehicle (EtOH:emulphor:saline=1:18) injections, or with the specific CB₁ receptor antagonist SR141716A (rimonabant)+vehicle. Injections were performed subcutaneously (s.c.) in the neck using 30 gauge needles (10 μg body weight), a second injection was performed in the flank. In order to minimize ‘litter effects’ (Fride and Weinstock, 1984), the various treatments were administered to the pups within each litter. Pups were examined daily during the first 8 days of life.

On each subsequent day, pups were briefly separated from their mothers for the duration of weighing and scoring of the presence of “milkbands” in their stomachs. (As the stomach area in mouse pups is transparent, due to lack of hair and the thinness of the skin, the amount of milk consumed can be observed as a “milkband”).

‘Nipple attachment’: A “foster” mother was anesthetized with an intraperitoneal (i.p.) injection of ketamine (100 mg/kg) and xylazine (20 mg/kg) and pups were observed for the strength of nipple attachment, scored as 0 (no holding onto nipple), 1 (weakly holding on to nipple) or 2 (firmly attached to nipple) (based on (Calamandrei and Valanzano, 1994; Wilson et al., 1981).

‘Lapping’: In one experiment, pups were also examined for their ability to ingest food by licking (‘lapping’) from a dish. In order to circumvent the need for oral-motor competence required for sucking milk through the maternal nipple, pups are placed in a dish in which a paper towel soaked with a mixture of milk (3%) and cream (28% fat), such that the pups are exposed to a uniform layer of liquid as described (Hall and Browde, 1986).

During testing, the mother was kept in a holding cage in a separate room. Pups were kept at an environmental temperature of 26-28° C.

Prepulse Inhibition (PPI) of the startle reflex: In this experimental model a weak stimulus (70-90 db tone) inhibits the subsequent response to a strong stimulus (120 db tone). Reduced prepulse inhibition of the startle reflex (PPI) is taken as an additional index of the positive symptoms of schizophrenia (Josselyn and Vaccarino, 1998; Swerdlow and Geyer, 1998). PPI was assessed as described previously (Varty et al., 2001). In the model employed, mice were placed in a startle chamber (Hamilton and Kinder, Poway, Calif., USA). A high frequency loudspeaker produces a 65 db background noise and the various acoustic stimuli: 77 and 81 db prepulse; 120 db startle, separated by 100 ms. The startle response of the mouse is transduced and stored by a computer. Sixty-five readings are taken at 1 ms intervals, and the average amplitude is taken as the startle response. Each test session lasts for about 20 min and consists of 10 presentations of each of the six trial types (Prepulse, Prepulse+Pulse, Pulse or no stimulus other than background) separated by 15 s. The amount of prepulse inhibition is calculated as %PPI=[1-(startle response for Prepulse+Pulse)/(startle response for Pulse alone)]×100.

Results: Rimonabant-treated pups from either Sabra or ICR strain did not hold on to the nipple and scored almost zero for nipple attachment (FIG. 4).

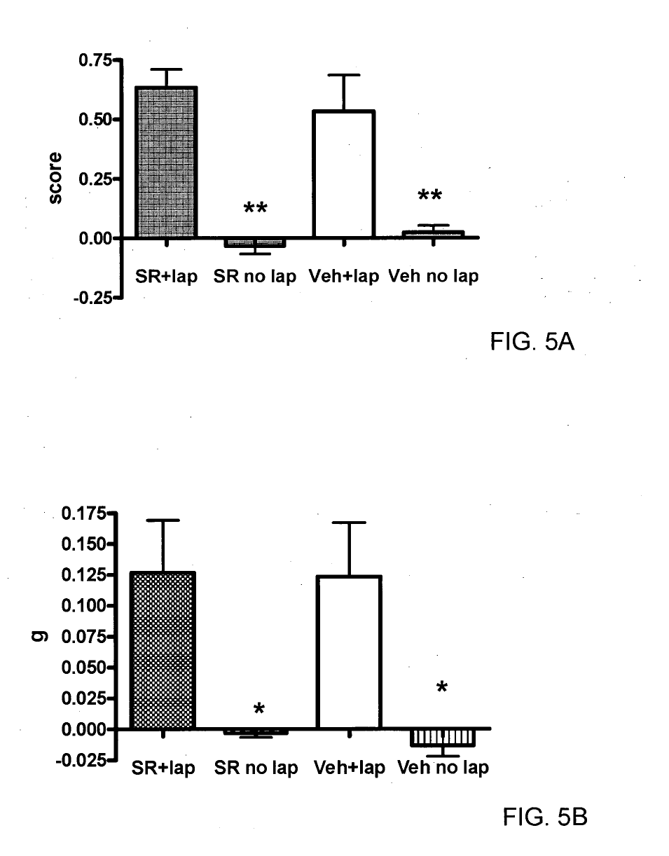

As can be seen in FIG. 5, when allowed to lick, rather than being forced to suck milk, food intake (A) and body weight gain (B) over the period of the exposure to the milk/cream mixture, were completely normalized in the CB₁ receptor-blocked pups.

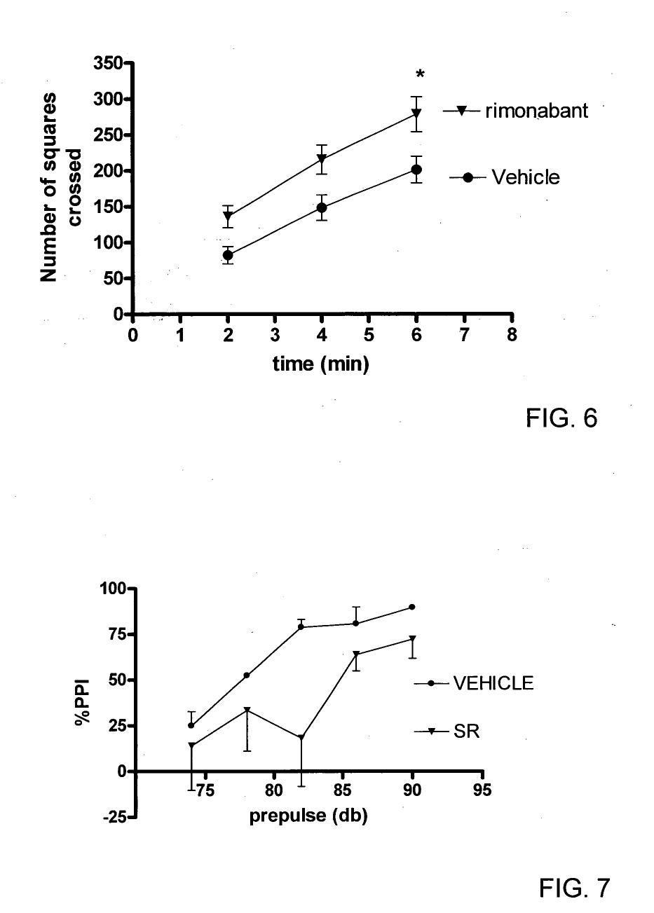

At 3 months of age, mice (ICR strain), treated with rimonabant at birth, were tested for motor activity (ambulation) in an open field. The bottom of the open field was divided into 20 squares of equal size. Activity was assessed as the number of squares crossed. As adults, pups exposed neonatally to rimonabant were hyperactive in the Open field (FIG. 6) (F=19.2, df=1, 12, P<0.001, 2-way ANOVA).

Finally, sensorimotor gating, measured as prepulse inhibition of an acoustic startle response (PPI), was significantly lower in rimonabant-treated pups compared to vehicle control (FIG. 7). This suggests that neonatal inhibition of CB_{I} receptors has permanent consequences for the maturing organism including hyperactivity and impaired sensorimotor gating. This is compatible with lingering cognitive deficiencies detected in adolescents who were diagnosed with NOFTT in infancy (Reifsnider, 1995).

### Example 3

### Intracerebral Versus Subcutaneous Injections of the CB1 Receptor Antagonist SR141716 (Rimonabant)

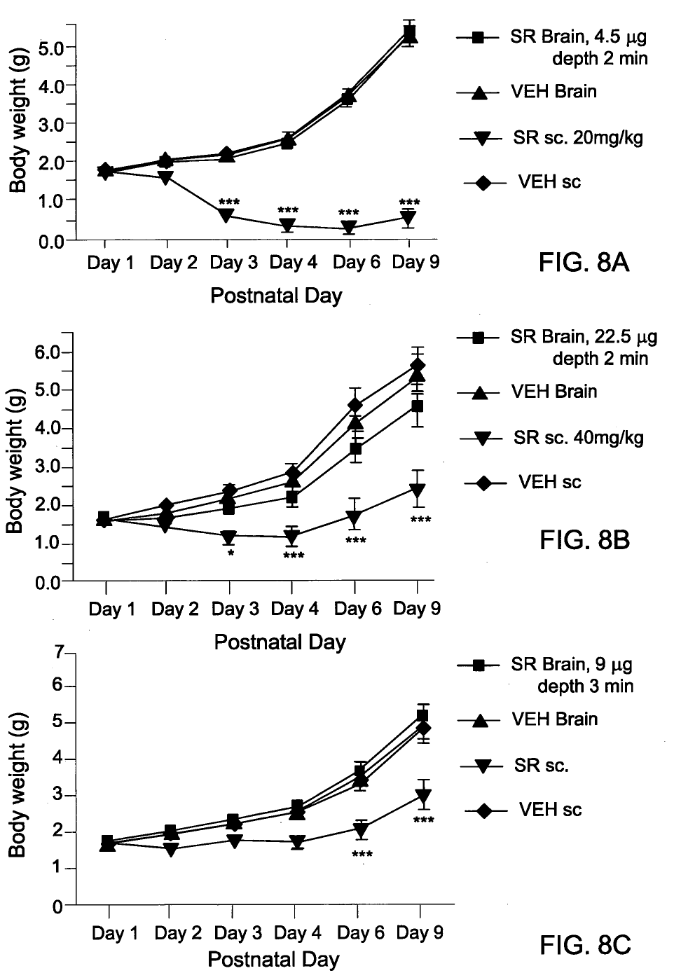

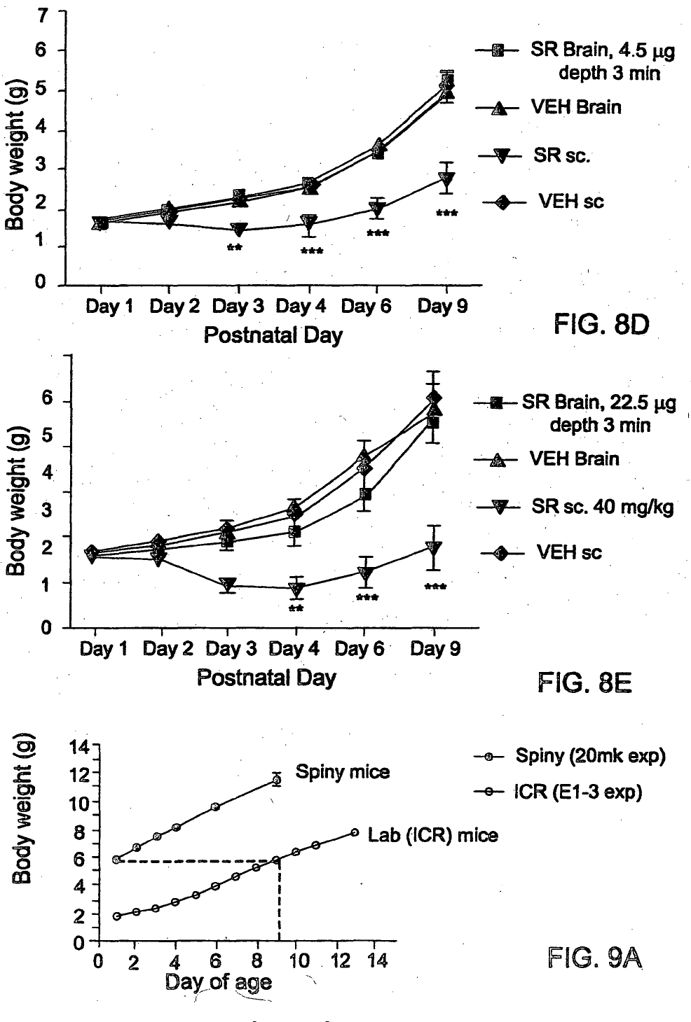

Newborn (day 1 of birth) mouse pups (ICR strain) were injected subcutaneously (sc) with SR141716 (SR, 20 or 40 mg/kg), as described previously (Fride, 2004; Fride et al., 2003; Fride et al., 2001). In addition, littermates were injected with SR directly into the brain, at 2 depths (2-3 mm) and at several doses 4.5, 9.0 or 22.5 μg (between 50 and 10-fold lower concentrations than the sc injections). As can be seen in FIG. 8A-E, none of the centrally injected regimens had any effect on pup growth, while the sc injections at both doses, in each experiment (FIGS. 8A-E), significantly retarded body weight gain.

These results may indicate a direct effect of the CB1 receptor antagonist on sucking-related nerves, and/or an effect on a digestive system-related mechanism. These tissues/organs were found to express CB1 receptors. These findings imply that infants with failure-to-thrive as a result of a deficient endocannabinoid system, may be treated with peripherally acting food additives or medications. For example, with peripherally restricted cannabinoid-based compounds such as those described U.S. Pat. No. 6,864,291 (Fride et al).

### Example 4

### Effect of Rimonabant on Spiny Wild Mice Newborn Development

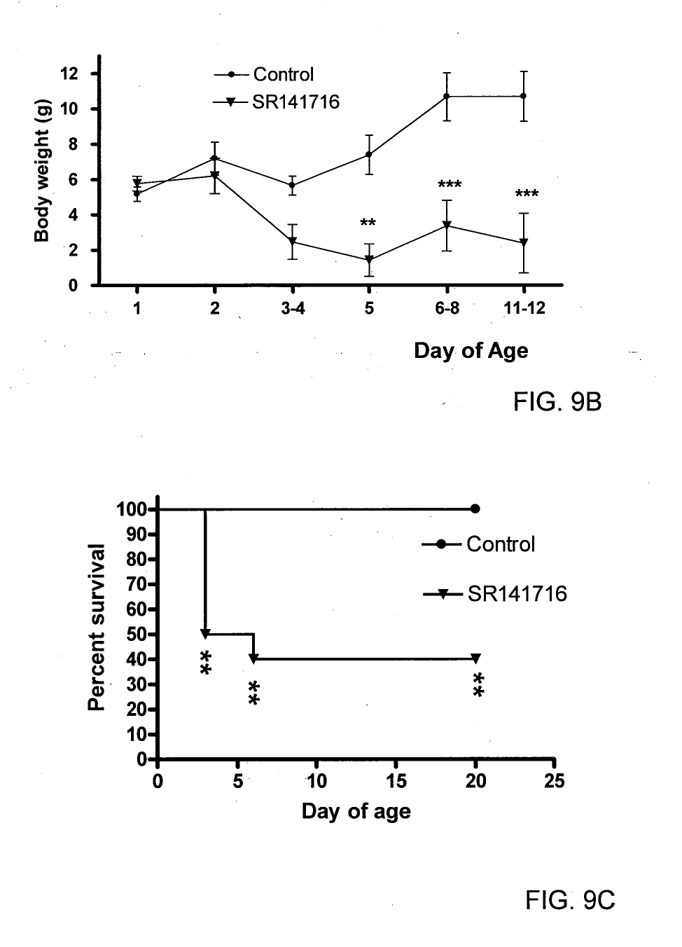

FIG. 9A illustrates the relative state of maturation in terms of the growth curve during the first two postnatal weeks in laboratory (ICR strain) mice, compared to Spiny wild mice (acomys cahirinus) which is born at a more advanced stage of development. As can be seen, on the day of birth of the spiny mice, the body weight is equivalent to that reached by ICR mice on postnatal day 9. This is similar to the difference in brain maturation predicted by the model designed by Clancy and colleagues (Clancy et al., 2007), i.e., the day of birth of the spiny mouse being equivalent to postnatal day 8 in the laboratory mouse.

When spiny mice were injected with 50 mg/kg rimonabant, a highly significant delay in growth (F=22.7, df 1,74, P<0.0001, FIG. 9B) is observed, due in part to the rimonabant-induced mortality. Mortality rates (60%) were significant in the rimonabant group compared to controls (Kaplan-Meier survival analysis: chi-square=7.6, df=1, P<0.01, FIG. 9C).

As noted above Spiny wild mice differ from laboratory mice in their relatively advanced development stage at birth. This implicates the importance of the endocannabinoid system in the postnatal development of both less developed newborns (e.g. ICR mice) and newborn with a more advanced development (e.g. Spiny mice). Such newborns, having a more advanced stage of maturation at birth, have a higher resemblance in their development stage at birth to human infants, thus emphasizing the importance of these findings for enhancing infant feeding, growth and development by administration of EC.

### REFERENCES

Calamandrei, G. and A. Valanzano, 1994, Age-dependent effects of NGF and scopolamine on suckling behavior of neonatal mice, Pharmacol Biochem Behav 49, 1043.
Fride, E., 2004, The Endocannabinoid-CB1 receptor system during gestation and postnatal development, European Journal of Pharmacology 500, 289.
Fride, E., C. Feigin, D. E. Ponde, A. Breuer, L. Hanus, N. Arshaysky and R. Mechoulam, 2004, (+)-Cannabidiol analogues which bind cannabinoid receptors but exert peripheral activity only, Eur J Pharmacol 506, 179.
Fride, E., A. Foox, E. Rosenberg, M. Faigenboim, V. Cohen, L. Barda, H. Blau and R. Mechoulam, 2003, Milk intake and survival in newborn cannabinoid CB1 receptor knockout mice: evidence for a “CB3” receptor, Eur J Pharmacol 461, 27.
Fride, E., Y. Ginzburg, A. Breuer, T. Bisogno, V. Di Marzo and R. Mechoulam, 2001, Critical role of the endogenous cannabinoid system in mouse pup suckling and growth, Eur J Pharmacol 419, 207.
Fride, E. and N. Gobshtis, 2007, Endocannabinoids and their Receptors: Physiology, Pathology and Pharmacology, Immunology, Endocrine and Metabolic agents in medicinal chemistry 7, 157.
Fride, E. and R. Mechoulam, 1993, Pharmacological activity of the cannabinoid receptor agonist, anandamide, a brain constituent, Eur J Pharmacol 231, 313.
Fride, E. and M. Weinstock, 1984, The effects of prenatal exposure to predictable or unpredictable stress on early development in the rat, Dev Psychobiol 17, 651.
Hall, W. G. and J. A. Browde, Jr., 1986, The ontogeny of independent ingestion in mice: or, why won't infant mice feed? Dev Psychobiol 19, 211.
Harkin, A., T. J. Connor, M. P. Burns and J. P. Kelly, 2004, Nitric oxide synthase inhibitors augment the effects of serotonin re-uptake inhibitors in the forced swimming test, Eur Neuropsychopharmacol 14, 274.
Josselyn, S. A. and F. J. Vaccarino, 1998, Preclinical behavioral approaches and study of antipsychotic drug action and schizophrenia, in: In vivo neuromethods, Vol. 32, eds. A. A. Boulton, G. B. Baker and A. N. Bateson (Humana Press, Totowa) p. 177.
Petit-Demouliere, B., F. Chenu and M. Bourin, 2005, Forced swimming test in mice: a review of antidepressant activity, Psychopharmacology (Berl) 177, 245.
Reifsnider, E., 1995, The use of human ecology and epidemiology in nonorganic failure to thrive, Public Health Nurs 12, 262.
Swerdlow, N. R. and M. A. Geyer, 1998, Using an animal model of deficient sensorimotor gating to study the pathophysiology and new treatments of schizophrenia, Schizophr Bull 24, 285.
Treit, D. and J. Menard, 1998, Animals models of anxiety and depression, in: In vivo Neuromethods, eds. A. Boulton, G. Baker and A. Bateson (Humana Press, Totowa) p. 89.
Varty, G. B., N. Walters, M. Cohen-Williams and G. J. Carey, 2001, Comparison of apomorphine, amphetamine and dizocilpine disruptions of prepulse inhibition in inbred and outbred mice strains, Eur J Pharmacol 424, 27.
Wilson, L. M., S. S. Chang, S. J. Henning and D. L. Margules, 1981, Suckling: developmental indicator of genetic obesity in mice, Dev Psychobiol 14, 67.

1. An infant formula comprising at least about 0.2 mg per liter liquid formula of an endocannabinoid.

2. The infant formula of claim 1, wherein the formula further comprises an endocannabinoid-promoting compound.

3. An infant formula comprising at least about 0.05 mg per liter liquid formula of an endocannabinoid promoting compound.

4. A pharmaceutical composition comprising as an active ingredient an endocannabinoid or an endocannabinoid promoting compound, or a combination thereof for promoting infant, child or adolescent feeding, growth or development.

5. An infant formula according claim 1, or a pharmaceutical composition according to claim 4 wherein the endocannabinoid is selected from the group consisting of anandamide, 2-arachidonoyl glycerol (2AG), noladin ether, N-arachidonoylglycerol dopamine (NADA) and virodhamine

6. An infant formula according to claim 2, or a pharmaceutical composition according to claim 4 wherein the endocannabinoid promoting compound is an inhibitor of a degrading enzyme or a reuptake inhibitor.

7. An infant formula according to claim 2, or a pharmaceutical composition according to claim 4 wherein the endocannabinoid promoting compound is a fatty acid glycerol ester.

8. An infant formula or a pharmaceutical composition according to claim 7 wherein the fatty acid glycerol ester is 2-palmytoyl glycerol or 2-linoleoyl-glycerol.

9. An infant formula according to claim 2, wherein said formula comprises at least about 0.05 mg endocannabinoid promoting compound per liter of liquid formula.

10. An infant formula according to claim 1 wherein the formula is a powder.

11. An infant formula according to claim 1 wherein the formula is a liquid.

12. An infant formula according to claim 1 or a pharmaceutical composition according to claim 4 wherein said endocannabinoid is a peripherally restricted cannabinoid-based compound.

13. An infant formula according to claim 1 for promoting infant feeding, growth or development.

14. An infant formula according to claim 13 wherein said infant suffers from Failure to thrive (FTT).

15. A method for enhancing infant feeding, growth or development comprising administering to an infant in need thereof a formula comprising an endocannabinoid in an amount sufficient to promote feeding, growth or development.

16. A method in accordance with claim 15 wherein said formula administered to the infant is sufficient to provide said infant with at least about 0.04 mg/kg/day of said endocannabinoid or wherein the infant consumes at least 0.2 mg/day of said endocannabinoid.

17. A method of claim 15 wherein said formula further comprises an endocannabinoid promoting compound.

18. A method of claim 15 wherein said infant suffers from Failure to thrive (FTT).

19. A method for promoting infant, child or adolescent feeding, growth or development comprising administering to an infant, a child or an adolescent in need thereof a pharmaceutical composition comprising an endocannabinoid in an amount sufficient to promote feeding, growth or development.

20. A method in accordance with claim 19 wherein said pharmaceutical composition further comprises an endocannabinoid promoting compound.

21. A method for promoting infant feeding, growth or development comprising administering to an infant in need thereof a formula comprising an endocannabinoid promoting compound in an amount sufficient to promote feeding, growth or development.

22. A method in accordance with claim 21 wherein said formula comprises at least about 0.05 mg per liter liquid formula of an endocannabinoid promoting compound.

23. A method in accordance with claim 21 wherein said infant suffers from Failure to thrive (FTT).

24. A method for promoting infant, child or adolescent feeding, growth or development comprising administering to an infant, a child or an adolescent in need thereof a pharmaceutical composition comprising an endocannabinoid promoting compound in an amount sufficient to promote feeding, growth or development.
